(19) United States
(12) Patent Application Publication
(10) Pub. No.: US 2014/0186466 A1
(43) Pub. Date: Jul. 3, 2014

(54) NEUTRACEUTICAL FORMULATION FOR TREATMENT OF DIABETES

(71) Applicant: Creative Medical Health Inc., Phoenix, AZ (US)

(72) Inventor: Amit Patel, Salt Lake City, UT (US)

(73) Assignee: Creative Medical Health Inc., Phoenix, AZ (US)

(21) Appl. No.: 14/144,753

(22) Filed: Dec. 31, 2013

Related U.S. Application Data
(60) Provisional application No. 61/747,616, filed on Dec. 31, 2012.

(51) Int. Cl. A61K 36/27; A61K 33/06; A61K 31/555; A61K 31/385; A61K 36/38; A61K 31/593; A61K 36/42; A61K 36/54; A61K 36/37; A61K 36/33; A61K 36/48; A61K 31/205; A61K 33/24; A61K 36/53

(52) U.S. Cl. CPC A61K 36/27; A61K 33/24; A61K 33/06; A61K 31/555; A61K 31/385; A61K 36/38; A61K 36/53; A61K 36/42; A61K 36/54; A61K 36/37; A61K 36/33; A61K 36/48; A61K 31/205; A61K 31/593
USPC 424/646; 424/682; 514/188; 514/440; 424/725; 424/745; 424/758; 424/739; 424/767; 424/757; 514/167

(57) ABSTRACT

Disclosed are compositions of matter useful for the treatment of diabetes. In one embodiment a nutraceutical composition is administered to a patient in need, said composition comprising of one or more ingredients selected from a group consisting of: magnesium, chromium picolinate, alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin D3.

### CROSS-REFERENCE TO RELATED APPLICATIONS

This application claims priority to Provisional Application Ser. No. 61/747,616, filed Dec. 31, 2012 and entitled “Neutraceutical Formulation for Treatment of Diabetes”, which is hereby expressly incorporated by reference in its entirety.

### FIELD OF THE INVENTION

The invention pertains to the field of diabetes. Specifically, the invention relates to the use of natural compounds for modulation of diabetes-associated insulin deficiency and/or resistance and diabetes-associated complications. More specifically, the invention relates to the use of combinations of Magnesium, Chromium Piccolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin d3.

### BACKGROUND

Diabetes (medically known as diabetes mellitus) refers to a group of diseases that are collectively characterized by abnormally high blood glucose due to impairment of the body's ability to produce or utilize glucose as an energy source. The imprecise regulation of blood glucose levels caused by the deficiency of insulin production and/or reliance upon injected insulin, culminate in a perpetual inflammatory state within the body that leads to multi-organ damage over the years. Accordingly, diabetes is associated with serious secondary complications due to these poorly controlled spikes in blood glucose. These complications include hypertension, heart disease and stroke, nervous system disease (neuropathy), critical limb ischemia, diabetic retinopathy (blindness), and renal failure. Therefore, diabetes has devastating effects on the health and quality of life of individuals with this condition. Approximately 18.8 million people in the United States have been diagnosed with diabetes, and it is estimated that an additional 7 million people remain undiagnosed (www.diabetes.org/diabetes-basics/diabetes-statistics, accessed Jul. 3, 2012). The health care costs associated with diabetes are enormous; for example, the total cost was estimated at $218 billion in the United States in 2007. Conservative estimates predict that the number of people with diabetes will more than double between 2000 and 2030.

There are several distinct forms of diabetes that confer similar symptoms and long-term complications, although the underlying causes of these disease variants differ. Type 1 diabetes, which is usually diagnosed by early adolescence, occurs due to autoimmune-mediated destruction of the pancreatic beta cells. Another form of diabetes has also been recognized, termed latent autoimmune diabetes (LADA) or type 1.5 diabetes, which is classified as a slowly progressing form of type 1 diabetes. Type 1 diabetes and LADA arise as a result of autoimmune attack on the insulin producing beta cells in the islets of Langerhans of the pancreas by self-reactive T lymphocytes. Extensive research has demonstrated that T cells that are reactive against beta cell antigens, such as insulin, are aberrantly expanded in individuals with recent-onset autoimmune diabetes as well as in subjects at-risk for the disease. At risk individuals are first-degree relatives of patients with type 1 diabetes, who may share specific susceptibility alleles in common, and patients exhibiting signs of pre-diabetes marked by humoral and/or cellular responses against insulin In patients with autoimmunity, islet-reactive T cells invade the islets where they target and destroy the beta cells in conjunction with other immune cell populations that propagate the inflammatory response. Autoimmune-mediated diabetes is believed to involve both genetic and environmental components, including infections with highly prevalent enteroviruses. Although the disease-initiating events in T1D are controversial and may vary between individuals, it is established that T cells are primary instigators of the transition from intra-islet inflammation to overt autoimmunity [1].

It is estimated that 1 of every 400 children or 250, 000 people under the age of 20 in the United States have been diagnosed with type 1 diabetes (http://www.diabetes.org/diabetes-basics/diabetes-statistics/, accessed: Jul. 4, 2012). These patients are reliant upon multiple daily insulin injections or continual infusion of insulin through a pump, and test their blood sugar by pricking their fingers for blood six or more times per day. Neither dietary therapy nor treatment with an oral hypoglycemic agent is effective, and only treatment with insulin is effective. Ketonemia and acidosis can occur due to the loss of insulin secreting capacity, and if untreated, may result in diabetic coma. Since numerous factors such as stress, hormones, growth, physical activity, medications, illness/infection, and fatigue effect insulin utilization, even a strictly monitored program of insulin administration does not mimic the endogenous functions of the pancreas, and numerous complications can develop over the lifetime of the patient.

Type 2 diabetes is an adult-onset disease resulting from inadequate insulin production or insulin resistance due to impairment of peripheral tissue response to insulin. Diabetes afflicts approximately 10.9 million people over the age of 65 in the United States, with the majority of these having type 2 diabetes (http://www.diabetes.org/diabetes-basics/diabetes-statistics/, accessed Jul. 4, 2012). However, as a result of the obesity epidemic, substantially younger patients are beginning to be diagnosed with this condition. Insulin resistance is present in almost all obese individuals [2]. However, compensatory insulin production by beta-cells usually occurs, thus preventing hyperglycemia. However, in response to prolonged insulin resistance, the pancreatic beta cells eventually lose their ability to cope with the increasing insulin demands and postprandial hyperglycemia occurs, characterizing the transition between normal glucose tolerance and abnormal glucose tolerance. Subsequently, the liver starts secreting glucose through hepatic gluconeogenesis (generation of glucose from substrates that are not sugars, not from glycogen) and hyperglycemia is observed even in the fasting state. In contrast to IDDM, NIDDM presents only a small degree of ketonemia and acidosis although the insulin action is reduced from normal, and treatment with insulin is not always required. The greatest clinical challenge in this disease is the prevention of the long-term complications, many of which involve vascular, ocular and renal systems. Although various agents are utilized to increase glucose sensitivity, insulin secretion, or exogenous insulin is used therapeutically, these do not exactly mimic the physiological control of post-prandial insulin secretion. Accordingly, the fluctuations of glucose, as well as downstream metabolic consequences lead to macrovascular pathology such as coronary atherosclerosis, and increased risk of stroke, as well as microvascular pathology such as macular degeneration and renal failure. Additionally, neuropathies are often present associated with hyperglycemia.

In the art, there are numerous treatments available for type 2 diabetes; these depend on patient-specific characteristics, as well as the severity of disease. The treatment goal in diabetes treatment is to bring plasma glucose levels down to as near normal levels, for example 80-120 milligrams per deciliter (mg/dl) before meals and 100-140 mg/dl at night. Numerous medical tests are known in the art for monitoring glucose, as well as cholesterol and lipid levels. The goal of maintaining normal glucose levels is judged in part by the ability to prevent secondary complications such as retinopathy, neuropathy, vascular disease, and strokes. In beginning phases of type 2 diabetes, patients may be treated with various oral drugs, and as diabetes progresses, various forms of insulin may be administered. Although tight glucose control is known to decrease the rate of diabetic complications, such control is very difficult to achieve, and when achieved significant morbidity and mortality still occurs. Mainstream oral treatments for diabetes can be separated by their mechanism of action into two groups: hypoglycemics, such as sulfonylureas and meglitinides which induce beta cell insulin secretion and antihyperglycemics such as biguanides and alpha-glucosidase inhibitors which cause uptake of glucose.

Sulfonylureas are a type of drug that stimulate insulin release from beta cells. Essentially, these agents work by blocking ATP-sensitive potassium channels in the pancreatic beta-cell membrane. This effect is mediated by the binding of the drug to the sulfonylurea receptor (SUR) subunit of the channel. Inhibition of the potassium channel leads to depolarization of the cell membrane and insulin secretion, in a similar way as if glucose was added to the cell. Glyburide is a second generation sulfonylurea compound that is sold under the names Micronase, DiaBeta, or Glynase. Glipizide, sold under the names Glucotrol and Glucotrol XL, is also a second generation sulfonylurea drug. Third-generation sulfonylurea drugs include Glimepiride (Amaryl). This agent is believed to have greater safety in patients with ischemic heart disease as compared to other sulfonylurea drugs. Glimepiride is the only sulfonylurea based drug that is approved for use together with insulin or metformin. In general, sulfonylurea drugs suffer from the disadvantage that the amount of insulin secretion induced depends on the timing and dose of drug administration and not by the blood glucose levels. This causes not only various fluctuations in glucose level but also digestive symptoms such as anorexia in some patients.

Meglitinides (commonly called glinides) are a class of insulin secretagogues that have short-acting activity, given after meals. Similar to sulfonylurea drugs in that mechanistically they induce insulin secretion by closure of the ATP-dependent potassium channel, glinides appear to be more short-term in activity. Theoretically these drugs have less risk of inducing hypoclycemia and cause a physiological-like insulin release pattern. Repaglinide, sold under the name Prandin, and Nateglinide, sold under the name Starlix, are examples of two glinides. When compared with sulfonylurea drugs, glinides have been shown to provide a better control of postprandial hyperglycaemia, not to induce hypoglycaemia, and to generally have better safety profile, especially in patients with renal failure [3]. Biguanides are a class of drugs that decrease hepatic glucose production and increase insulin sensitivity. Metformin, sold under the names Glucophage, Glucophage XR, and Metformin XR is an example of a biguanide. It is also the most widely prescribed oral antidiabetic in the world and is in most circumstances the agent of choice for first line initial therapy of the typical obese patient with type 2 DM and mild to moderate hyperglycaemia [4]. Metformin administration is associated with weight loss and improvement in lipid profile. Metformin is effective as monotherapy and, in combination with both insulin secretagogues and thiazolidinediones (TZDs), may alleviate the need for insulin treatment [5]. It is known that metformin induces increased glucose utilization and reduction in leptin concentrations [6]. Additionally, metformin induces inhibition of dipeptidyl peptidase-IV activity, which allows for extended half-life of GLP-1 [7]. Classical mechanisms of action include increased glucose use by anaerobic glycolysis, inhibition of hepatic gluconeogenesis, and suppression of intestinal absorption of glucose. One adverse effect associated with various biguanides is lactic acidosis. Thiazolidinediones (glitazones) are a family of drugs that decrease insulin resistance in both muscle and adipose tissue. They do not induce insulin secretion. Rosiglitazone, sold under the name Avandia, and Pioglitazone, sold under the name Actos are two thiazolidinediones. These agents induce insulin sensitivity through the activation of insulin receptor kinase, thereby promoting glucose uptake by peripheral tissues, and ameliorating increased liver glucose production. Known side effects include digestive symptoms and edema, and hematological alterations, and upregulation in plasma LDH. Glitazones are interesting not only from their ability to increase insulin signal transduction, but also due to anti-inflammatory effects. It is known, for example, that rosiglitazone inhibits ability of dendritic cells to secrete interleukin-12 after stimulation via CD40 [8]. This is believed to occur via activation of PPAR-gamma pathways. Additionally, treatment with rosiglitazone is able to inhibit onset of colitis in animal models through preferential induction of Th2 cytokine production [9]. Alpha-glucosidase inhibitors are used to delay rate of sugar absorption. Acarbose, sold under the name Precose, and Miglitol sold under the name Glyset are two examples of drugs in this family. Incretin mimetics mirror glucose-dependent insulin secretion, cause inhibition of glucagon secretion, and delay gastric emptying. Exenatide, sold under the name Byetta, is a glucagon-like-peptide-1 (GLP-1) receptor agonist and stimulates insulin secretion from the beta cell. Controlled clinical trials provided evidence that glycaemic control under exenatide administered twice daily in a dose of 5-10 microg was not inferior to conventional insulin therapy.

In the art, numerous approaches to control the development of diabetes-related complications have been investigated to target the diverse cellular and molecular mechanisms that underlie these pathologies. As known in the art, attempts are being made to harness the regenerative capacity of various stem/progenitor cell populations in order to directly generate new beta cells or to address the diabetes-associated inflammatory complications. These regenerative cell types include “stem cells”, known to those skilled in the art as being pluripotent or totipotent undifferentiated cells capable of continuous renewal, or “progenitor cells” that are derived from stem cells and are committed toward a particular cell lineage. Islet transplants have allowed patients with Type 1 diabetes to discontinue insulin injections for 1-5 years; however the widespread use of this treatment is limited by insufficient availability of islets from cadaveric donors, inadequate assessments of viability of isolated islets, and the side effects resulting from the reliance upon immune suppressive drugs. As is known in the art, a major thrust of diabetes research is harnessing the properties of stem cells/progenitor to generate an adequate supply of insulin-secreting beta cells and/or to modulate the pro-inflammatory disease mechanisms.

A cell population demonstrated in the art to possess regenerative potential is the endothelial progenitor cell (EPC). EPC are bone marrow-derived cells that are responsible for self-renewal and health of the vasculature through their ability to differentiate directly into endothelial cells. There is tight regulation of mobilization of EPC from the bone marrow into the circulation; stimulatory factors (eg. statins used to treat coronary artery disease) and inhibitory factors (cardiovascular risk factors) modulate EPC levels, thereby affecting the body's vascular repair capacity. Indeed, studies have shown that, in patients with cardiovascular risk factors, the number of EPCs that can be isolated from peripheral blood is reduced and EPC function is impaired. An inverse correlation exists between the number of EPCs and the subjects' combined Framingham risk factor score [10]. Measurements of flow-mediated brachial-artery reactivity also show a significant correlation between endothelial function and the number of EPCs, supporting a role for EPCs in maintaining endothelial integrity. Endothelial damage represents a balance between the extent of injury and the capacity for repair. In diabetes, hyperglycemia is associated with endothelial cell dysfunction and reduced neovascularization in response to tissue damage. High glucose concentrations that typify diabetes inhibit EPC growth. Patients with type 2 diabetes exhibit reduced numbers of circulating EPC [11]. Research shows that EPC are required to promote healing of diabetic ulcers and endothelial repair. Therefore, one possible therapeutic intervention for diabetes is to administer agents, either pharmacological or natural, which could bolster the endogenous numbers or functions of EPC.

### SUMMARY

Embodiments herein relate to methods of ameliorating the effects of diabetes in a mammal comprising: identifying a mammal suffering from diabetes; administering at least 2 or more naturally occurring substances selected from the group consisting of: magnesium, chromium picolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin d3 in an amount sufficient to ameliorate the effects of diabetes in said mammal.

Further embodiments relate to A method to increase the efficacy of allogeneic or an autologous stem cells delivered therapeutically to a subject suffering from diabetes, said method comprising: identifying a subject suffering from diabetes, administering said stem cell therapy to said subject together with a composition containing one or more ingredients selected from the group consisting of: Magnesium, Chromium Piccolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin D3.

### DETAILED DESCRIPTION

The invention teaches compositions of herbs, vitamins and natural products for control and/or prevention of diabetes and its complications. In some embodiments, the formulation is used as a monotherapy, in other embodiments, one of skill in the art utilizes said formulation in conjunction with treatments known to decrease insulin resistance, to affect the disease process or to address the complications associated with diabetes. In other embodiments of the invention, said formulations are used together with monitoring of circulating endothelial progenitor cells as a means of guiding the need for administration of said formulation, as well as the frequency of administration required. In other embodiments of the invention, said formulations are used in conjunction with specific markers of autoimmunity, inflammation and/or obesity as a means for guiding the need for administration of said formulation.

In one embodiment of the invention, the nutraceutical composition described herein is utilized as an adjunct to stem cell therapy. Numerous types of stem cell therapies exist and are known in the art that may be utilized in combination with the current invention. For example, said nutraceuticals can be used in combination with autologous bone marrow stem cell therapy, as currently practiced using devices such as the Harvest, Arteriocyte, Tissue Genesis, or Bio-met device. Another example is the use of said nutraceutical composition in combination with agents known in the art to mobilize endothelial progenitor cells (EPC), which can be performed by administering said composition with G-CSF and natural remedies such as green tea and resveratrol.

One or more ingredients comprising the invention described herein can be administered in beverages, tonics, infusions, or food-stuffs alone, or in combination with other dietary supplements or therapeutics. The herb-based composition of the present invention can be administered alone or formulated with pharmaceutically acceptable compounds known in the art, vehicles, or adjuvants with a favorable delivery profile, i.e., suitable for delivery to a subject. A “pharmaceutically acceptable carrier” can include any solvent(s), dispersion media, coatings, antibacterial and antifungal compounds, isotonic and absorption delaying compounds that are compatible with pharmaceutical administration. Preferred examples of “pharmaceutically acceptable carriers” include, but are not limited to, water, saline, Ringer's solutions, dextrose solution, and 5% human serum albumin. The composition may also be formulated as a suspension, such as a liposomal suspension, where liposomes and non-aqueous vehicles such as fixed oils are used, or formulated in an aqueous emulsion. A composition of the present invention may be formulated to be compatible with its intended route of administration. Examples of routes of administration for components of the present invention include oral, buccal, injectable, intravenous, intraperitoneal, subcutaneous, inhalational, intramuscular, intraarticular, intraarterial, intracerebral, intracerebellar, intrabronchial, intrathecal, parenteral, rectal, sublingual, topical, transdermal, and aerosol route. Oral compositions generally include an inert diluent or an edible carrier. They can be enclosed in gelatin capsules, caplets compressed into tablets, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, gummie, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, yogurts, cereal coatings, foods, nutritive foods, and combinations thereof. The composition of the present inventive subject matter may be administered in a partial, i.e., fractional dose, one or more times during a 24 hour period, a single dose during a 24 hour period of time, than a double dose during a 24 hour period of time, or more an a double dose during a 24 hour period of time. Fractional, double or other multiple doses may be taken simultaneously or at different times during the 24 hour period.

In one embodiment, magnesium is administered as magnesium sulfate at a concentration sufficient to alter diabetic processes, and/or inhibit progression of diabetic secondary complications. One of skill in the art is referred to Heidarianpour et al. [12] who sought to determine whether chronic magnesium sulfate administration could control streptozocin-induced diabetes and improve endothelium-dependent and endothelium-independent dilatation, and identify its probable mechanism in the skin microvasculature of diabetic rats. Fifty male Wistar rats were separated (220±10 g) into 3 groups: 2 were induced to develop diabetes by treatment with the beta cell toxin streptozotocin, and one served as a control group. One of the diabetic subgroups was given magnesium sulfate (10 g/l) in their drinking water, while two other groups where given only tap water. Stimulation of endothelial relaxation with acetylcholine (Ach) or sodium nitroprusside (SNP) was enhanced in the group that received magnesium sulfate (0.1 M). Through the use of a nitric oxide inhibitor, it was demonstrated that magnesium improvement of skin microvasculature circulation in the diabetic rats was associated with stimulation of the nitric oxide pathway. Hence, the invention may be practiced together with agents that simulate the nitric oxide pathway.

In another study, rats were segregated into 4 groups: one group served as control, while diabetes was induced in the other groups with a single i.v. injection of 40 mg/kg streptozotocin [13]. Magnesium-treated chronic diabetic rats received 10 g/l of magnesium sulfate added to the drinking water (0.46 g/24 h) for eight weeks. An increase in plasma glucose, high density lipoprotein (HDL), low density lipoprotein (LDL), very low density lipoprotein (VLDL), total cholesterol and triglyceride concentrations was observed in the diabetic animals. Reductions in plasma Mg were observed to correlate with diabetes. Administration of magnesium sulfate for eight weeks reduced plasma glucose, high density lipoprotein (HDL), low density lipoprotein (LDL), very low density lipoprotein (VLDL), total cholesterol and triglyceride to normal levels. Moreover, magnesium supplementation led to reductions in the mesenteric fat/body weight ratio and returned systolic blood pressure to normal levels. Additionally, Soltani et al. studied the effects of magnesium administration on vascular complications associated with diabetes [14]. The animals were divided into a group that received tap water for 8 weeks, and rats that were made diabetic with a single IV injection of 40 mg/kg STZ and either treated with magnesium sulfate (10 g/L) added to the drinking water or given tap water only. The three groups of animals (control, diabetic and Mg-treated), had their mesenteric vascular beds perfused according to the McGregor method, and descending thoracic aortas were used for measurement of elasticity. Baseline perfusion pressure of diabetic group was significantly higher than control and Mg-treated groups with intact and denuded endothelium. Magnesium sulfate treatment was found to decrease the mean perfusion pressure of mesenteric vascular bed in intact and denuded endothelium.

In studies of patients with type 2 diabetes, low dietary magnesium intake is associated with metabolic syndromes and depression. Central obesity, high body fat percentage, and high body mass index are significantly lower with increasing quantities of magnesium intake. Epidemiologic and clinical data have revealed that a high magnesium diet (at least 500-1000 mg/d) lowers blood pressure [15, 16]. In a study of 60 patients with hypertension given magnesium oxide at 20 mmol/d during 8 weeks, significant reductions in ambulatory, home, and office BP were observed [17]. The office BP fell by 3.7/1.7 mm Hg, ambulatory BP was reduced by 2.5/1.4 mm Hg, and home BP decreased by 2.0/1.4 mm Hg. Importantly also, the serum and urinary magnesium concentrations correlated with the BP reduction. Patients with the highest BP levels exhibited the largest reduction in BP in response to dietary magnesium.

In one embodiment, chromium piccolinate is administered along with said composition of ingredients comprising Magnesium, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin D3. In the art, the trace element chromium is known to be essential for carbohydrate metabolism. Chromium picolinate has been reported to potentiate the action of insulin and to lower plasma triglyceride and cholesterol levels when administered to diabetic rats at 1 mg/kg body weight for a period of 4 weeks [18]. Streptozotocin-induced diabetic animals were administered chromium as chromium picolinate at a daily dose of 1 mg/kg body weight for a period of 4 weeks. It was observed that chromium picolinate lowered plasma glucose levels, and increased weight loss in diabetic animals. Chromium also normalized glycogen content in the livers of diabetic animals to near control levels. Another effect of chromium picolinate is reducing the activity of inflammatory pathways involved in diabetic nephropathy, as shown in a rat model. In this study, rats were fed either a normal diet consisting of 8% fat or a high fat diet of 40% fat and chromium picolinate was dissolved in water and administered to the animals at 22 micrograms for 12 weeks (providing 8 mcg/day), which is equivalent to 560 micrograms for a 70 kg adult human [19]. Chromium picolinate treatment as 10 mg chromium/kg diet for six weeks was associated with recovery of myocardial contractility and relaxation following ischemia-reperfusion insult in rats [20]. According to these findings, chromium picolinate can be utilized to address the complications associated with diabetes. Chromium levels were lower in the liver of diabetic rats when compared with that of control rats. A negative correlation was observed between plasma glucose and chromium concentration in patients with diabetes [18]. The data suggests that chromium picolinate supplementation is beneficial in correcting hyperglycemia.

In a study where chromium picolinate was administered to diabetic rats at at low (human equivalent) and high doses (2.90 and 13.20 μg Cr kg (−1) day (−1), respectively, elevated levels of hepatic and cerebral free fatty acids and malondialdehyde were significantly reduced by low doses of chromium picolinate and were nearly normalized to control (non-diabetic) levels in the high dose group [21].

In humans, chromium picolinate (1000 mug/day) therapy was shown to improve insulin resistance in HIV-positive subjects. Eight subjects on antiretroviral therapy were treated with chromium picolinate for eight weeks and insulin sensitivity was measured with a hyperinsulinaemic-euglycaemic insulin clamp. The mean rate of glucose disposal was measured to be 4.41 mg glucose/kg lean body mass (LBM)/min (range 2.67-5.50), which increased to 6.51 mg/kg LBM/min (range 3.19-12.78, p=0.03), an increase of 25% following chromium picolinate treatment.

In one embodiment, alpha lipoic acid is administered along with said composition of ingredients comprising magnesium, chromium picolinate, Garcina indica, holy basil, Morodica charantica, cinnamon, Salacia reticulate, Salacia oblonga, Gymnema sylvestre, nopal cactus, fenu greek, vanadium, 1-carnitine, and vitamin D3. Alpha lipoic acid is a fatty acid found naturally in every cell and is needed for the conversion of glucose into energy. Alpha lipoic acid is considered an antioxidant that neutralizes free radicals, which are waste products created when the body turns food into energy. Alpha lipoic acid is made by the body and can be found in very small amounts in foods such as spinach, broccoli, peas, Brewer's yeast, brussel sprouts, rice bran, and organ meats such as liver.

In the art, alpha lipoic acid is known to lower blood sugar in patients with diabetes and in animal models of disease. In one report, 57 patients with type 2 diabetes were assigned to receive either ALA (300 mg daily) or placebo [22]. Eight weeks later, patients fasted overnight and 2 hours after breakfast, blood samples were drawn. Fasting blood glucose levels were reduced by alpha lipoic acid treatment. Additionally, administration of alpha lipoic acid reduced measures of platelet reactivity in a study of 51 type 1 diabetic patients to whom alpha lipoic acid was administered (600 mg once daily) for 5 weeks [23]. Synthetic alpha lipoic acid is commercially available as a nutritional supplement and has been shown to be effective at ameliorating symptoms in diseases with an underlying oxidative stress component through reductions in LDL cholesterol and triglycerides, and ameliorating the oxidative susceptibility of lipoproteins [24].

In one embodiment, alpha lipoic acid is administered intravenously along with said composition of ingredients comprising magnesium, chromium picolinate, Garcina indica, holy basil, Morodica charantica, cinnamon, Salacia reticulate, Salacia oblonga, Gymnema sylvestre, nopal cactus, fenu greek, vanadium, 1-carnitine, and vitamin D3 that are administered via other routes, such as orally. In the art, alpha lipoic acid supplements are available in capsule form. Additionally, alpha-lipoic acid has been administered intravenously for treatment of peripheral neuropathy. When given intravenously at a dosage of 600 mg/day over a period of 3 weeks, alpha lipoic acid induces a clinically relevant reduction in neuropathic pain, whereas it is not clear whether oral administration of>600 mg/day can provide symptomatic relief for patient [25].

In one embodiment, garcinia indica is administered along with said composition of ingredients comprising magnesium, chromium picolinate, alpha lipoic acid, holy basil, Morodica charantica, cinnamon, Salacia reticulate, Salacia oblonga, Gymnema sylvestre, nopal cactus, fenu greek, vanadium, 1-carnitine, and vitamin D3. Garcinia indica belongs to Clusiaceae family, and is a slowly growing polygamodioecious tree. It is distributed through out topical Asia, Africa and Polynesia, and in the topical humid evergreen rain forest of Western Ghats of South India and the North Eastern states of India. Garcinol, a polyisoprenylated benzophenonethe compound extracted from the fruit rind, is known in the art to have anti-inflammatory and neuroprotective properties. Garcinol has been demonstrated to promote expansion of stem cells ex vivo; therefore, this compound is a candidate for bolstering the levels of therapeutic stem cells. Garcinol was also shown to up-regulated the gene expression of adiponectin as well as down-regulated the gene expressions of leptin and Fas, indicative of its anti-adipogenic effects for treating obesity-related conditions [26]. When garcinia indica was administered orally to diabetic rats as an aqueous extract at 100-200 mg/kg for 4 weeks, the fasting and postprandial blood glucose levels were significantly reduced [27]. Hence, garcinia indica and its components have utility for addressing diabetes and its associated complications.

In one embodiment, holy basil is administered along with said composition of ingredients comprising magnesium, chromium picolinate, alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin D3. Holy basil (Ocimum tenuiflorum) is found in liquid capsule and liquid extract form and is a source of phytochemicals with strong antioxidant, anti-inflammatory, and stress-reducing properties. In a randomized, placebo-controlled, single blind trial, holy basil leaves were administered to patients with type 2 diabetes, the levels of fasting and postprandial blood glucose were reduced by 17.6% and 7.3%, respectively [28].

In one embodiment, Morodica charantica is administered along with said composition of ingredients comprising magnesium, chromium picolinate, alpha lipoic acid, Garcina indica, holy basil, cinnamon, Salacia reticulate, Salacia oblonga, Gymnema sylvestre, nopal cactus, fenu greek, vanadium, 1-carnitine, and vitamin D3. Morondica charantica, commonly known as bitter melon (BM), is recognized as a remedy for diabetes and its complications, particularly in India, Southeast Asia, Africa and South American. Animal studies indicate that BM juice is effective in regulating weight gain, likely due to its effects as a potent inhibitor of adipocyte differentiation. In a dose response study, oral glucose tolerance was improved in rats fed a high fat (HF; 30%) diet supplemented with freeze-dried BM juice at a dose of 0.75% or higher [29]. At the highest dose, BM-supplemented rats tended to have less visceral fat mass. In another study where rats were fed a high-fat diet supplemented without or with 5% lyophilized BM powder, the BM treatment prevented hyperinsulinaemia and glucose intolerance, and this treatment was associated with reduced expression of lipogenic genes [30]. Hence, morondica charantica influences glucose and lipid metabolism in a manner that is favorable for addressing diabetes and its related complications.

In one embodiment, cinnamon is administered along with said composition of ingredients comprising magnesium, chromium picolinate, alpha lipoic acid, Garcina indica, holy basil, Morodica charantica, Salacia reticulate, Salacia oblonga, Gymnema sylvestre, nopal cactus, fenu greek, vanadium, 1-carnitine, and vitamin D3. Cinnamon is recognized as having hypoglycemic effects with potentially beneficial effects on blood glucose control in patients with prediabetes and diabetes. In a randomized, double-blind study, 66 patients with type 2 diabetes were assigned to groups that received either placebo or low-dose or high-dose supplementation with cinnamon extract at 120 and 360 mg/d, respectively. Patients in all 3 groups took the anti-diabetic drug gliclazide during the 3 month study. Fasting blood glucose and glycosylated hemoglobin A(1c) levels were significantly reduced in patients in the low- and high-dose groups as compared to the placebo group. In a study evaluating the literature on the effects of cinnamon on clinical and biochemical parameters, Cinnamomum zeylanicum was found to attenuate diabetes-associated weight loss, reduce fasting blood glucose, LDL and HbA(1c), increase HDL cholesterol, and increase circulating insulin levels [31]. Additionally, cinnamon had benefits against the complications of diabetes such as diabetic neuropathy and nephropathy.

In one embodiment, salacia reticulata is administered along with said composition of ingredients comprising magnesium, chromium picolinate, alpha lipoic acid, Garcina indica, holy basil, Morodica charantica, cinnamon, Salacia oblonga, Gymnema sylvestre, nopal cactus, fenu greek, vanadium, 1-carnitine, and vitamin D3. Salacia reticulata, a Hippocrateaceae plant that grows in India and Sri Lanka, has been used traditionally in Ayurvedic medicine to treat diabetes and as a food supplement to prevent diabetes and obesity. Salacia reticulata has protective properties against metabolic diseases including suppression of body weight increase and fat accumulation, alleviation of abnormal lipid metabolism and abnormal glucose tolerance, and suppression of intrahepatic fat accumulation in a mouse model of type 2 diabetes [32]. The anti-obesity mechanism of salacia reticulata involves inhibition of adipocyte differentiation [33].

In one embodiment, Salacia oblonga is administered along with said composition of ingredients comprising magnesium, chromium picolinate, alpha lipoic acid, Garcina indica, holy basil, Morodica charantica, cinnamon, salacia reticulata, Gymnema sylvestre, nopal cactus, fenu greek, vanadium, 1-carnitine, and vitamin D3. The Salacia oblonga plant has been used extensively in Ayurvedic medicine to maintain healthy blood sugar levels. Salacia oblonga contains two alpha-Glucosidase inhibitors: salicinol and kotalanol 9, which impede the body's absorption of carbohydrates. In a randomized study of 66 patients with type 2 diabetes in a fasted state, subjects consumed 1 of the following meals: a standard liquid control meal, a control meal+240 mg Salacia oblonga extract, and a control meal+480 mg Salacia oblonga extract [34]. The results showed that Salacia oblonga lowers acute glycemia and insulinemia resulting from a high carbohydrate meal.

In one embodiment, Gymnema sylvestre is administered along with said composition of ingredients comprising magnesium, chromium picolinate, alpha lipoic acid, Garcina indica, holy basil, Morodica charantica, cinnamon, Salacia oblonga, salacia reticulata, nopal cactus, fenu greek, vanadium, 1-carnitine, and vitamin D3. Gymnema sylvestre is an herb native to tropical forests of India that has long been recognized as having anti-diabetic properties. The active ingredient, gymnemic acid, is extracted from the leaves and roots, and is involved in balancing blood glucose by binding to glucose receptors in the lining of the intestines, thereby inhibiting glucose uptake and preventing the elevation of blood glucose levels.

In one embodiment, nopal cactus is administered along with said composition of ingredients comprising magnesium, chromium picolinate, alpha lipoic acid, Garcina indica, holy basil, Morodica charantica, cinnamon, Salacia oblonga, salacia reticulata, Gymnema sylvestre, fenu greek, vanadium, 1-carnitine, and vitamin D3. Nopal cactus, also known as prickly pear cactus, is the most commonly used treatment for lowering blood sugar in type 2 diabetes among individuals of Mexican descent [35]. It is known in the art that nopal cactus can be administered as a tea, added into food, or in capsule form. One study reported the addition of nopal to tortillas led to lowered blood glucose levels as well as lower cholesterol and triglycerides in the plasma of healthy volunteers [36].

In one embodiment, fenu greek is administered along with said composition of ingredients comprising magnesium, chromium picolinate, alpha lipoic acid, Garcina indica, holy basil, Morodica charantica, cinnamon, Salacia oblonga, salacia reticulata, Gymnema sylvestre, nopal cactus, vanadium, 1-carnitine, and vitamin D3. Fenugreek, which means “ram's horn clover,” is an herb with numerous medicinal properties that grows in the Mediterranean regions of the world. The seeds of fenugreek can be sprinkled on prepared food or taken in capsule form. Fenugreek is believed to lower blood glucose levels by slowing the absorption of carbohydrates due to its high fiber content. In a clinical study of fenugreek, subjects with Type 1 diabetes were treated with fenugreek seed powder (100 g) divided into two equal doses as part of their lunch and dinner. Dietary fenugreek was found to significantly reduce fasting blood glucose and also resulted in a 54% reduction in 24-hour urinary glucose excretion [37]. Lowered blood cholesterol (total and LDL) was also observed in individuals that received dietary fenugreek. Hence, fenugreek is utilized as a dietary supplement with benefits for managing diabetes.

In one embodiment, vanadium is administered along with said composition of ingredients comprising magnesium, chromium picolinate, alpha lipoic acid, Garcina indica, holy basil, Morodica charantica, cinnamon, Salacia oblonga, salacia reticulata, Gymnema sylvestre, nopal cactus, fenu greek, 1-carnitine, and vitamin D3. There is experimental evidence that supplementation with vanadium can be beneficial for addressing diabetes and diabetic complications. In one study, diabetes was chemically induced in rats with streptozotocin and subjected to cerebral artery occlusion, followed by treatment with sodium orthovanadate (0.6 mg/mL) in their drinking water for 4 weeks [38]. The results showed that vanadium significantly reduced the ischemic injury in diabetic rat brains, as evidenced by improved neurobehavioral function. In another study, diabetic rats were treated with vanadyl sulfate (100 mg/kg) for 60 days, which was shown to lower the blood glucose levels and decrease serum antioxidant enzyme levels in muscle tissue [39].

In one embodiment, 1-carnitine is administered along with said composition of ingredients comprising magnesium, chromium picolinate, alpha lipoic acid, Garcina indica, holy basil, Morodica charantica, cinnamon, Salacia oblonga, salacia reticulata, Gymnema sylvestre, nopal cactus, fenu greek, vanadium, and vitamin D3. L-carnitine is a naturally occurring amino acid that is produced in the body from two amino acids, methionine and lysine and is present in meats, dairy products, and avocados. Thus, carnitine concentrations in the body are maintained through synthesis of L-carnitine in the body, dietary L-carnitine, and elimination and reabsorption of carnitine by the kidneys. The functions of 1-carnitine in the body include facilitating the transport of long-chain fatty acids across the mitochondrial membrane, thereby participating in beta oxidation of fatty acids [40]. Moreover, fatty acid transport across mitochondrial membranes also leads to reduced cytosolic accumulation of triglycerides, which improves insulin sensitivity. Indeed, administration of acetyl-L-carnitine shows dose-dependent effects on tissue uptake of glucose in healthy patients and in patients with type 2 diabetes [40]. There is research in support of 1-carnitine deficiency in patients with diabetes [41, 42]. Acetyl-1-carnitine administration reportedly provides a degree of relief from diabetes-related complications. In support of this concept, patients with diabetes were treated with 500 mg or 1000 mg acetyl-1-carnitine three times a day for 52 weeks. This study reported symptomatic relief from neuropathic pain, as well as improvements in nerve fiber regeneration [43]. L-carnitine supplementation also reportedly improves the effects of the anti-obesity drug Orlistat. Patients who took L-carnitine combined with Orlistat showed better improvement in body weight, inflammatory markers and glucose and lipid profiles as compared to subjects who took Orlistat alone [44, 45].

In one embodiment, vitamin D3 is administered along with said composition of ingredients comprising magnesium, chromium picolinate, alpha lipoic acid, Garcina indica, holy basil, Morodica charantica, cinnamon, Salacia oblonga, salacia reticulata, Gymnema sylvestre, nopal cactus, fenu greek, vanadium, 1-carnitine. Vitamin D3 (2000 IU/daily for 18 months) has been shown to have beneficial effects on serum lipid profiles in patients with diabetes [46]. It is known in the art that there are epidemiological links between vitamin D deficiency, diabetes mellitus, and diabetes-related complications including dyslipidemia and coronary artery disease.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### REFERENCES

1. Horwitz, M. S., et al., Requirements for viral-mediated autoimmune diabetes: beta-cell damage and immune infiltration. J Autoimmun, 2001. 16(3): p. 211-7.
2. Paoletti, R., et al., Metabolic syndrome, inflammation and atherosclerosis. Vasc Health Risk Manag, 2006. 2(2): p. 145-52.
3. Scheen, A. J., Drug-drug and food-drug pharmacokinetic interactions with new insulinotropic agents repaglinide and nateglinide. Clin Pharmacokinet, 2007. 46(2): p. 93-108.
4. Joshi, S. R., Metformin: old wine in new bottle—evolving technology and therapy in diabetes. J Assoc Physicians India, 2005. 53: p. 963-72.
5. Goodarzi, M. O. and M. Bryer-Ash, Metformin revisited: re-evaluation of its properties and role in the pharmacopoeia of modern antidiabetic agents. Diabetes Obes Metab, 2005. 7(6): p. 654-65.
6. Eriksson, A., et al., Short-term effects of metformin in type 2 diabetes. Diabetes Obes Metab, 2007. 9(3): p. 330-6.
7. Green, B. D., et al., Inhibition of dipeptidyl peptidase-IV activity by metformin enhances the antidiabetic effects of glucagon-like peptide-1. Eur J Pharmacol, 2006. 547(1-3): p. 192-9.
8. Faveeuw, C., et al., Peroxisome proliferator-activated receptor gamma activators inhibit interleukin-12 production in murine dendritic cells. FEBS Lett, 2000. 486(3): p. 261-6.
9. Saubermann, L. J., et al., Peroxisome proliferator-activated receptor gamma agonist ligands stimulate a Th2 cytokine response and prevent acute colitis. Inflamm Bowel Dis, 2002. 8(5): p. 330-9.
10. Xiao, Q., et al., Endothelial progenitor cells, cardiovascular risk factors, cytokine levels and atherosclerosis—results from a large population-based study. PLoS One, 2007. 2(10): p. e975.
11. van Ark, J., et al., Type 2 diabetes mellitus is associated with an imbalance in circulating endothelial and smooth muscle progenitor cell numbers. Diabetologia, 2012. 55(9): p. 2501-12.
12. Heidarianpour, A., et al., The influence of oral magnesium sulfate on skin microvasculature blood flow in diabetic rats. Biol Trace Elem Res, 2011. 143(1): p. 344-50.
13. Soltani, N., M. Keshavarz, and A. R. Dehpour, Effect of oral magnesium sulfate administration on blood pressure and lipid profile in streptozocin diabetic rat. Eur J Pharmacol, 2007. 560(2-3): p. 201-5.
14. Soltani, N., et al., Effects of administration of oral magnesium on plasma glucose and pathological changes in the aorta and pancreas of diabetic rats. Clin Exp Pharmacol Physiol, 2005. 32(8): p. 604-10.
15. Jee, S. H., et al., The effect of magnesium supplementation on blood pressure: a meta-analysis of randomized clinical trials. Am J Hypertens, 2002. 15(8): p. 691-6.
16. Touyz, R. M., Role of magnesium in the pathogenesis of hypertension. Mol Aspects Med, 2003. 24(1-3): p. 107-36.
17. Kawano, Y., et al., Effects of magnesium supplementation in hypertensive patients: assessment by office, home, and ambulatory blood pressures. Hypertension, 1998. 32(2): p. 260-5.
18. Sundaram, B., K. Singhal, and R. Sandhir, Ameliorating effect of chromium administration on hepatic glucose metabolism in streptozotocin-induced experimental diabetes. Biofactors, 2012. 38(1): p. 59-68.
19. Selcuk, M. Y., et al., Chromium picolinate and chromium histidinate protects against renal dysfunction by modulation of NF-kappaB pathway in high-fat diet fed and Streptozotocin-induced diabetic rats. Nutr Metab (Lond), 2012. 9: p. 30.
20. Abebe, W., et al., Effects of chromium picolinate on vascular reactivity and cardiac ischemia-reperfusion injury in spontaneously hypertensive rats. Pharmacol Rep, 2010. 62(4): p. 674-82.
21. Refaie, F. M., et al., Effect of chromium supplementation on the diabetes induced-oxidative stress in liver and brain of adult rats. Biometals, 2009. 22(6): p. 1075-87.
22. Ansar, H., et al., Effect of alpha-lipoic acid on blood glucose, insulin resistance and glutathione peroxidase of type 2 diabetic patients. Saudi Med J, 2011. 32(6): p. 584-8.
23. Mollo, R., et al., Effect of alpha-lipoic acid on platelet reactivity in type 1 diabetic patients. Diabetes Care, 2012. 35(2): p. 196-7.
24. Harding, S. V., T. C. Rideout, and P. J. Jones, Evidence for using alpha-lipoic acid in reducing lipoprotein and inflammatory related atherosclerotic risk. J Diet Suppl, 2012. 9(2): p. 116-27.
25. Mijnhout, G. S., et al., Alpha lipoic Acid for symptomatic peripheral neuropathy in patients with diabetes: a meta-analysis of randomized controlled trials. Int J Endocrinol, 2012. 2012: p. 456279.
26. Hsu, C. L., et al., Inhibitory effects of garcinol and pterostilbene on cell proliferation and adipogenesis in 3T3-L1 cells. Food Funct, 2012. 3(1): p. 49-57.
27. Kirana, H. and B. Srinivasan, Aqueous extract of garcinia indica choisy restores glutathione in type 2 diabetic rats. J Young Pharm, 2010. 2(3): p. 265-8.
28. Agrawal, P., V. Rai, and R. B. Singh, Randomized placebo-controlled, single blind trial of holy basil leaves in patients with noninsulin-dependent diabetes mellitus. Int J Clin Pharmacol Ther, 1996. 34(9): p. 406-9.
29. Chen, Q., L. L. Chan, and E. T. Li, Bitter melon (Momordica charantia) reduces adiposity, lowers serum insulin and normalizes glucose tolerance in rats fed a high fat diet. J Nutr, 2003. 133(4): p. 1088-93.
30. Huang, H. L., et al., Bitter melon (Momordica charantia L.) inhibits adipocyte hypertrophy and down regulates lipogenic gene expression in adipose tissue of diet-induced obese rats. Br J Nutr, 2008. 99(2): p. 230-9.
31. Ranasinghe, P., et al., Efficacy and safety of ‘true’ cinnamon (Cinnamomum zeylanicum) as a pharmaceutical agent in diabetes: a systematic review and meta-analysis. Diabet Med, 2012.
32. Shimada, T., et al., Metabolic disease prevention and suppression of fat accumulation by Salacia reticulata. J Nat Med, 2010. 64(3): p. 266-74.
33. Shimada, T., et al., Salacia reticulata inhibits differentiation of 3T3-L1 adipocytes. J Ethnopharmacol, 2011. 136(1): p. 67-74.
34. Williams, J. A., et al., Extract of Salacia oblonga lowers acute glycemia in patients with type 2 diabetes. Am J Clin Nutr, 2007. 86(1): p. 124-30.
35. Shapiro, K. and W. C. Gong, Natural products used for diabetes. J Am Pharm Assoc (Wash), 2002. 42(2): p. 217-26.
36. Guevara-Arauza, J. C., et al., Biofunctional activity of tortillas and bars enhanced with nopal. Preliminary assessment of functional effect after intake on the oxidative status in healthy volunteers. Chem Cent J, 2011. 5(1): p. 10.
37. Sharma, R. D., T. C. Raghuram, and N. S. Rao, Effect of fenugreek seeds on blood glucose and serum lipids in type I diabetes. Eur J Clin Nutr, 1990. 44(4): p. 301-6.
38. Liu, Z., et al., Protection by vanadium, a contemporary treatment approach to both diabetes and focal cerebral ischemia in rats. Biol Trace Elem Res, 2012. 145(1): p. 66-70.
39. Kurt, O., et al., Influence of vanadium supplementation on oxidative stress factors in the muscle of STZ-diabetic rats. Biometals, 2011. 24(5): p. 943-9.
40. Mingrone, G., Carnitine in type 2 diabetes. Ann N Y Acad Sci, 2004. 1033: p. 99-107.
41. Scarpini, E., et al., L-carnitine and acetyl-L-carnitine in human nerves from normal and diabetic subjects. J Peripher Nerv Syst, 1996. 1(2): p. 157-63.
42. Ido, Y., et al., Neural dysfunction and metabolic imbalances in diabetic rats. Prevention by acetyl-L-carnitine. Diabetes, 1994. 43(12): p. 1469-77.
43. Sima, A. A., et al., Acetyl-L-carnitine improves pain, nerve regeneration, and vibratory perception in patients with chronic diabetic neuropathy: an analysis of two randomized placebo-controlled trials. Diabetes Care, 2005. 28(1): p. 89-94.
44. Derosa, G., et al., Comparison between orlistat plus 1-carnitine and orlistat alone on inflammation parameters in obese diabetic patients. Fundam Clin Pharmacol, 2011. 25(5): p. 642-51.
45. Derosa, G., et al., Orlistat and L-carnitine compared to orlistat alone on insulin resistance in obese diabetic patients. Endocr J, 2010. 57(9): p. 777-86.
46. Al-Daghri, N. M., et al., Vitamin D supplementation as an adjuvant therapy for patients with T2DM: an 18-month prospective interventional study. Cardiovasc Diabetol, 2012. 11(1): p. 85.

What is claimed is:

1. A method of ameliorating the effects of diabetes in a mammal comprising: identifying a mammal suffering from diabetes; administering at least 2 or more naturally occurring substances selected from the group consisting of: magnesium, chromium picolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin d3 in an amount sufficient to ameliorate the effects of diabetes in said mammal.

2. The method of claim 1, wherein said combination of naturally occurring substances is selected from the group consisting of: Magnesium at a concentration between 8 mg-8000 mg, Chromium Piccolinate at a concentration between 100 mcg-10000 mcg, Alpha lipoic acid at a concentration between 50 mg-5000 mg, Garcina indica at a concentration between 200 mg-20000 mg, holy basil at a concentration between 50-5000 mg, Morodica charantica at a concentration between 50 mg-5000 mg, cinnamon at a concentration between 100 mg-10000 mg, Salacia reticulate at a concentration between 40 mg-4000 mg, Salacia oblonga at a concentration between 50 mg-5000 mg, Gymnema sylvestre at a concentration between 40 mg-4000 mg, nopal cactus at a concentration between 100 mg-10000 mg, fenu greek at a concentration between 60 mg-6000 mg, vanadium at a concentration between 10 mcg-1000 mcg, 1-carnitine at a concentration between 50 mg-5000 mg, and vitamin D 3 at a concentration between 100 IU-10,000 IU.

3. The method of claim 1, wherein said combination of naturally occurring substances is selected from a group consisting of: Magnesium at approximately 800 mg, Chromium Piccolinate at approximately 1000 mcg, Alpha lipoic acid at approximately 500 mg, Garcina indica at approximately 2000 mg, at approximately holy basil 500 mg, Morodica charantica at approximately 500 mg, cinnamon at approximately 1000 mg, Salacia reticulate at approximately 400 mg, Salacia oblonga at approximately 500 mg, Gymnema sylvestre at approximately 400 mg, nopal cactus at approximately 1000 mg, fenu greek at approximately 600 mg, vanadium at approximately 100 mcg, 1-carnitine at approximately 500 mg, vitamin d3 at approximately 1000 IU.

4. A method of treating individuals at-risk for diabetes or having pre-diabetes comprising: identifying an individual at-risk for diabetes or having pre-diabetes, administering a combination of a group of naturally occurring substances selected from the group consisting of: Magnesium, Chromium Piccolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin d3.

5. The method of claim 4, wherein said combination of naturally occurring substances is selected from a group comprising of: Magnesium at a concentration between 8 mg-8000 mg, Chromium Piccolinate at a concentration between 100 mcg-10000 mcg, Alpha lipoic acid at a concentration between 50 mg-5000, Garcina indica at a concentration between 200 mg-20000 mg, holy basil at a concentration between 50-5000 mg, Morodica charantica at a concentration between 50 mg-5000 mg, cinnamon at a concentration between 100 mg-10000 mg, Salacia reticulate at a concentration between 40 mg-4000 mg, Salacia oblonga at a concentration between 50 mg-5000 mg, Gymnema sylvestre at a concentration between 40 mg-4000 mg, nopal cactus at a concentration between 100 mg-10000 mg, fenu greek at a concentration between 60 mg to 6000 mg, vanadium at a concentration between 10 mcg-1000 mcg, 1-carnitine at a concentration between 50 mg-5000 mg, and vitamin D 3 at a concentration between 100 IU-10,000 IU.

6. The method of claim 4, wherein factors dictating diabetes risk and/or pre-diabetes include one or more of the following: a) Circulating T cells with specificity for islet autoantigens; b) Circulating beta cell-specific autoantibodies; c) Obesity; d) Hypertension; e) Elevated fasting blood glucose levels (100-125 mg/dl); f) High blood glucose measured in the oral glucose tolerance test; g) High triglycerides; h) Family history of diabetes; i) Previous gestational diabetes.

7. The method of claim 1, wherein assessment of the dose of said combination needed is performed by measuring blood glucose, or by measuring A1c hemoglobin.

8. The method of claim 1, wherein assessment of the dose of said combination needed is performed by assessment of said patient endothelial reactivity.

9. The method of claim 8, wherein said assessment of patient endothelial reactivity is performed using the flow mediated dilation assay.

10. The method of claim 8, wherein said assessment of patient endothelial reactivity is performed to ascertain reactivity in a nitric oxide dependent or independent manner.

11. The method of claim 1, wherein assessment of the dose of said combination needed is determined by quantification of the levels of endothelial progenitor cells in the blood.

12. The method of claim 11, wherein said endothelial progenitor cells are detected by an agent capable of binding a molecule selected from the group consisting of: a) CD34; b) CD133; c) KDR-1; and d) CD166.

13. The method of claim 12, wherein said endothelial progenitor cells are detected by ability of forming endothelial cells when cultured in liquid culture, said endothelial cells expressing ability to uptake acetylated LDL.

14. The method of claim 1, wherein the dose of said composition needed is determined by measurement of advanced glycation end products (AGEs), their precursors, receptors for advanced glycation end products (RAGEs), or reactive oxygen species in blood and tissues of individuals to whom said composition is administered.

15. The method of claim 1, wherein assessment of the dose of said combination needed is determined by quantifying cytokines produced by peripheral blood mononuclear cells from individuals treated with said composition.

16. The method of claim 15, wherein the cytokines include one or more of the following; a) IFN-γ; b) IL-17; c) TNF-α; d) TGF-β (3; e) IL-10; f) IL-2

17. The method of claim 1, wherein assessment of the dose of said combination needed is determined by measuring the reactivity of circulating T cells against pancreatic antigens cultured with said T cells in protein or peptide form.

18. The method of claim 17, wherein the pancreatic antigens are components of the following proteins; a) Insulin; b) Proinsulin; c) GAD65; d) GAD67; e) tyrosine phosphatase IA-2; f) Chromogranin A; g) zinc transporter 8; h) ICA69.

19. The method of claim 1, wherein assessment of the dose of said combination needed is performed by quantification of immune regulatory/anti-inflammatory T cells in the circulation.

20. The method of claim 19, wherein said regulatory T cells are detected by an agent capable of binding a molecule selected from the group of: a) forkhead box protein 3 (FoxP3); b) CD25; c) TGF-β; d) glucocorticoid-induced TNFR-related protein (GITR); d) CTLA-4.

21. The method of claim 1, wherein assessment of the dose of said combination needed is performed based on measurements of adiposity selected from the group consisting of: a) Body mass index; b) Waist circumference; c) Percent body fat; d) Leptin levels.

22. The method of claim 1, wherein assessment of the dose of said combination needed is performed by evaluation of acute phase inflammatory proteins in the blood selected from the group consisting of: a) fibrinogen; b) IL-6; and c) high sensitivity C-reactive protein.

23. A method to increase the efficacy of a stem/progenitor cell mobilizing agent in a subject suffering from diabetes comprising: identifying a subject suffering from diabetes, administering said mobilizing agent to said subject together with a composition containing one or more ingredients selected from a group consisting of: Magnesium, Chromium Piccolinate, Alpha lipoic acid, Garcina indica, holy basil, Morodica charantica, cinnamon, Salacia reticulate, Salacia oblonga, Gymnema sylvestre, nopal cactus, fenu greek, vanadium, 1-carnitine, and vitamin D3.
